(19) United States
(12) Patent Application Publication
(10) Pub. No.: US 2011/0129870 A1
(43) Pub. Date: Jun. 2, 2011

(54) Microbial Screen Test

(76) Inventor: Peter E. Rising, Brightwaters, NY (US)

(21) Appl. No.: 12/674,574

(22) PCT Filed: Aug. 21, 2008
(86) PCT No.: PCT/US08/73885

§ 371 Date: Feb. 22, 2010

Related U.S. Application Data
(60) Provisional application No. 60/957,028, filed on Aug. 21, 2007.

(51) Int. Cl. C12Q 1/24

(52) U.S. Cl. 435/30

(57) ABSTRACT

A method for testing a microbial population including a negative screen includes inoculating a plurality of test ampoules with respective samples and recording a time of the inoculation (102a), performing a first light transmittance test on the test ampoules (103a), recording first test data, performing a second light transmittance test on the test ampoules (106a), recording second test data, and detecting negative samples based on the first test data and the second test data and the time of inoculation (107a).

### CROSS-REFERENCE TO RELATED APPLICATION

This application claims priority to U.S. Provisional Application Ser. No. 60/957,028, filed on Aug. 21, 2007, which is herein incorporated by reference in its entirety.

### BACKGROUND OF THE INVENTION

1. Technical Field

The present invention relates to a system and method for detecting and analyzing microbial activity.

2. Description of Related Art

The ASM (American Society for Microbiology) recommends that microbial samples be tested within 2 hours of sampling to maintain representative characteristics of the sample. It is the current practice in the field of urinary tract infection (UTI) analysis to analyze samples after 12-24 hours of sample storage. While precautions are taken to minimize microbial specie balance and concentration levels changes, industry studies demonstrate that sample representativeness is significantly compromised. Hence, the number of false positives and false negatives due to contamination and time are significant in the over prescribing of treatment drugs, unneeded hospital stays and general time insensitive test results for medical staff (e.g., MDs).

### SUMMARY OF THE INVENTION

According to an embodiment of the present disclosure, a method for testing a microbial population including a negative screen includes inoculating a plurality of test ampoules with respective samples, performing a first light transmittance test on the test ampoules, recording first test data, performing a second light transmittance test on the test ampoules, recording second test data, detecting negative samples based on the first test data and the second test data, and reporting negative samples based on the first test data and the second test data.

According to an embodiment of the present disclosure, a method for testing a microbial population including a false positive screen includes inoculating a plurality of test ampoules with respective samples, performing a first light transmittance test on the test ampoules, recording first test data, performing a second light transmittance test on the test ampoules, recording second test data, detecting samples positive for the presence of the microbial population based on the first test data and the second test data, and reporting the samples positive for the presence of the microbial population based on the first test data and the second test data.

### BRIEF DESCRIPTIONS OF THE DRAWINGS

Preferred embodiments of the present disclosure will be described below in more detail, with reference to the accompanying drawings:

FIG. 1 is a flow chart of a testing method including a negative screen according to an embodiment of the present disclosure;

FIG. 2 is a flow chart of a testing method including a negative screen for an implementation using remote testing according to an embodiment of the present disclosure;

FIG. 3 is a graph of performance characteristics of exemplary implementations according to embodiments of the present disclosure;

FIGS. 4A-B are graphs of performance pre-incubation characteristics for performing screen tests according to an exemplary implementations according to embodiments of the present disclosure;

FIGS. 5A-B are graphs of negative screen characteristics of exemplary implementations according to embodiments of the present disclosure;

FIG. 6 is a flow charts of a method for analyzing an aqueous sample according to an embodiment of the present disclosure; and

FIG. 7 is a diagram of a computer system for implementing a method according to an embodiment of the present disclosure.

### DETAILED DESCRIPTION OF PREFERRED EMBODIMENTS

According to an embodiment of the present disclosure, a liquid sample can be analyzed for the presence and activity of a biologic component by determining the transmittance of light through the sample, wherein the transmittance visible light is indicative of respiration and infrared light is indicative of a chemical reaction (e.g., reduction due to a TTC indicator). Multiple tests can be performed on the same sample at different times to determine growth characteristics of the sample. Further, according to an embodiment of the present disclosure, false negative results and false positive results may be identified for individual samples. In a first test or read determinations (e.g., positive/negative for the presence of bacteria) are made based on predetermined histograms based on physical properties of the sample for visible and IR, log/lag phase determinations in time to concentrations analysis, and microbe identification. In a subsequent test or read log/lag phases determinations may be confirmed, microbes identified, and false positive and negative samples (as identified in the first test) can be identified.

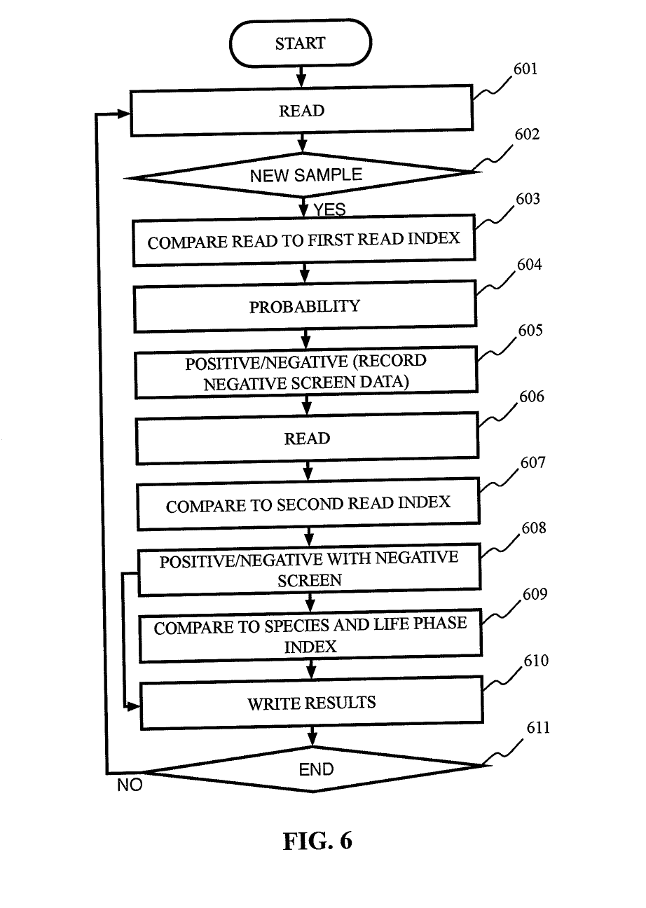

A spectrophotometer is used to read and record light transmission through an aqueous sample, where measures are recorded in a test record. The sample is taken and wavelengths are selected for first read analysis, these wavelengths for testing are available through the spectrophotometer having different light sources. A determination of potentially positive samples may be made using the first read analysis. The samples, e.g., potential positive samples, may be incubated and a second read is performed for each wavelength of the first read. A change in light transmission through the sample over time is determined. For example, if an increase in absorbance and/or a decrease in transmittance in a visible wavelength and an IR wavelength is determined, than the sample is confirmed to be positive. According to an embodiment of the present disclosure, negative samples may be determined during the first read analysis and discarded. Further, by comparing the curves for light transmission over time with known curves for a given species, a species of the sample can be determined and or the susceptibility to various anti microbial agents can be determined. FIG. 6 is a flow chart of an exemplary method for testing samples.

For example, a human urine analysis for 106 microbial concentration using 580 nm and 800 nm at 2 hours of incubation is considered positive if the 580 nm drops 20% T (transmission rate) or more and the 800 nm reading drops 10% T or more. With the predetermined spectral change information, the sample may be withdrawn from incubation and read spectrophotometrically a second time. The spectral output change is then compared to the predetermined values for change to be classified positive or negative. If a change in light transmittance satisfies a known value for a positive sample, the sample is considered positive and in the log phase of growth at time of sampling. If a change in light transmittance satisfies a known value of a negative sample, the sample is considered negative for the light wavelengths being tested and any bacteria present are in lag phase.

In this context, a negative screen system may be implemented which uses a collection of data points to reduce a number of potential false positives and to improve the use of scarce bio-laboratory resources.

According to an embodiment of the present disclosure, the act of inoculating a test ampoule is a start of the test. In conventional methods a test is not begun until a growth plate is streaked in a laboratory environment, leading to aging of the sample—samples are typically considered not to be representative if they are older than about 2-3 hours without refrigeration to stop bacterial growth.

The test ampoule is a controlled testing environment. Sample representativeness is ensured through the inoculation and simultaneous test start. The first read can therefore be done at the time of inoculation (e.g., within about 2-3 hours of inoculation) or later if the sample is refrigerated. By ensuring sample representativeness the probability of false positive and false negative results can be reduced. According to an exemplary embodiment, in the case of urine testing (UTI), by substantially eliminated false results, doctors can more accurately prescribe medication and avoid using medication for well patients.

According to an embodiment of the present disclosure, a test system can be expanded to include a satellite system in communication with a central system, without loss of bio-expertise or control. A single test system may be used to perform the methods described herein.

A satellite system may be used to first capture midstream urine sample microbiology characteristics at the time of inoculation (e.g., within about 30 minutes of inoculation). Testing of old urine samples, e.g., 8-20 hours, is a reason for inflated positive UTI reporting rates and a group of false negatives called “Mixed Contaminants” in prior systems.

According to an embodiment of the present disclosure, test inoculation for urine UTI culturing may be performed in the field (e.g., at the test site). The satellite system includes a computer system including a database and code for the elimination of potential false positives.

The satellite system may be based on the configuration and protocol as described below. At the satellite collection laboratory, a test system may be installed including the following components: reader, single read non-incubating reader or multi reading with auto incubation (e.g., spectrophotometer); database (including known characteristics of different microbes) and computer system, which may include video conferencing capabilities; bar code reader for use with labeled test ampoules and software for tracking the ampoules over multiple tests; and test ampoules. The test ampoules may be a sealed container having a negative pressure therein for drawing a predetermined volume of liquid into the ampoule. The test ampoules may further include a reagent dosed into the sealed container.

For small samples (e.g., pediatric samples), a sample may be diluted, e.g., up to about 1:9, to make adequate sample available throughout the process. Computer software can auto adjust all test criteria for first and second read and incubation time.

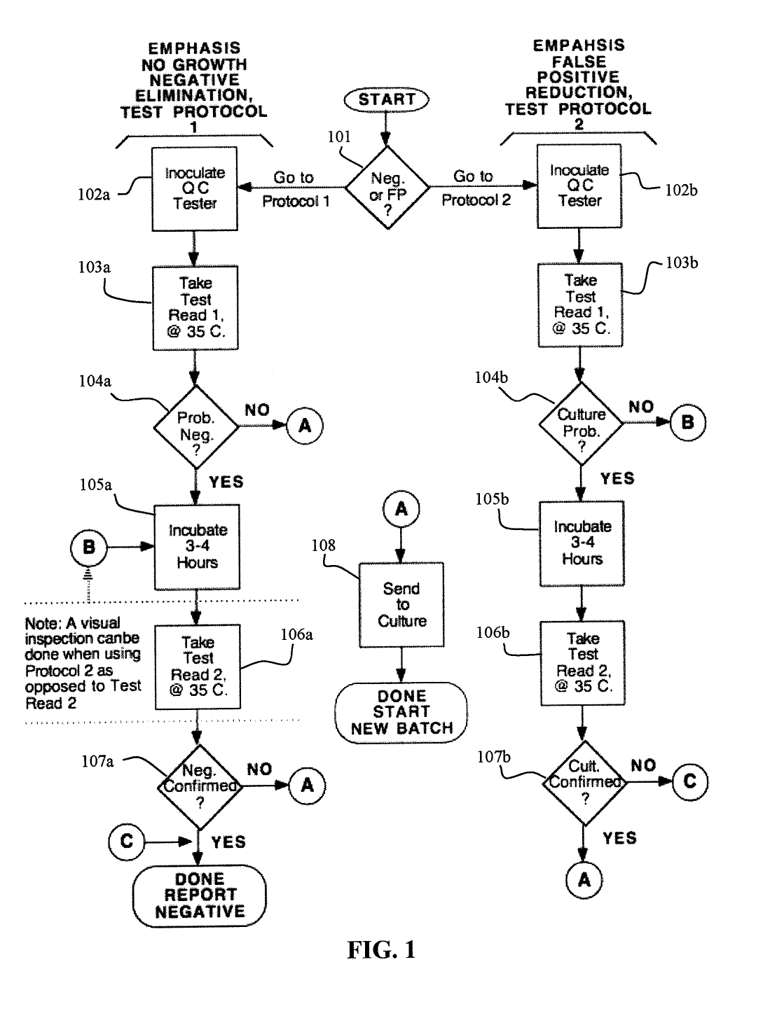

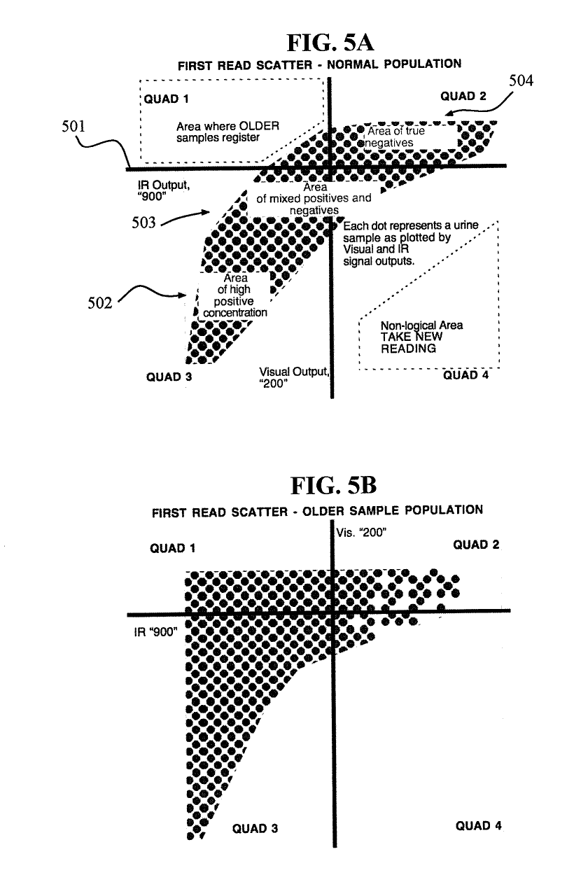

FIG. 1 is an exemplary satellite collection site protocol. The protocol may be performed within about 2-6 hours of inoculating a test ampoule. At block 101, a negative or a false positive test can be performed. Block 101 illustrates that negative or false positive determinations can be made, however the determination of the type of test is performed at blocks 104a and 104b based on the first read at blocks 103a and 103b. For example, in FIG. 5A samples having certain light transmittance characteristics determined at blocks 103a and 103b are identified as belonging to an areas of likely positive 502, mixed positives and negatives 503, and likely negative 504, wherein a false positive analysis can be performed for samples in a predetermined region of the area of high positive concentration and mixed positives and negative and a negative screen analysis can be performed for samples in a different predetermined region of the area of true negatives and mixed positives and negative. These regions can be determined from historical data and may vary with bacterial species. At blocks 102a-b, the inoculated test ampoule is inserted into a reader for test-read 1 at blocks 103a-b. Each test ampoule can be tracked, e.g., using a scan by the system bar code reader for associating an ampoule's code with a patient identifier. At blocks 104a-b, the database program designates the sample for processing to the central laboratory (e.g., probable positive) or retention at the satellite site, e.g., for 24 hours, before disposal (e.g., probable negative). At the end of each sample collection cycle, the database electronically transmits testing data for the past collection cycle to the database located at the central laboratory. The transmitted records from the satellite lab act as a sample control ticket for samples being sent at block 108, and contain test data for probable negative samples not sent as well as the culture probable samples. Embedded in the transmitted data are the time and date of each test-read 1. At blocks 105a-b, all of the samples retained at the satellite collection laboratory are incubated for about 2-4 hours and a test-read 2 is taken (Negative Screen Protocol 1) at blocks 106a-b. At blocks 107a-b the results test-read 2 checks the results of blocks 104a-b. Samples demonstrating bio-activity are sent to the central laboratory on the next shipping cycle. The entire control of satellite laboratory software may be from the central laboratory. Additionally, all satellite labs may have a video conference link to the central laboratory for training and sample processing questions. Interpretive results listed by the modified database program may be presented at the satellite laboratory and/or central laboratory. The satellite collection laboratory system may be designed to satisfy CLIA (Clinical Laboratory Improvement Amendments) protocols.

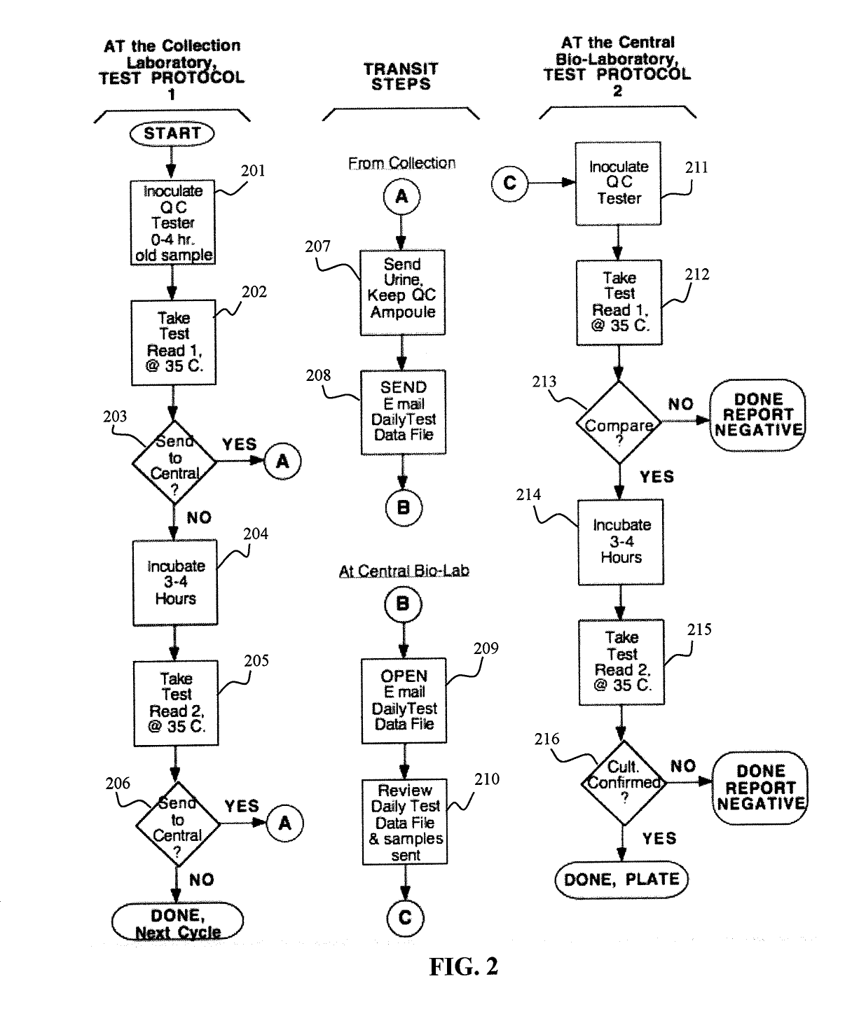

FIG. 2 depicts an exemplary process flow for a satellite utilization protocol. At block 201a test ampoule is inoculated and an initial test is performed at block 202 within about 0-4 hours of inoculation. At block 203 a determination is made whether to send the test ampoule to a central laboratory, for example, upon determining the sample of the test ampoule meets a criteria of the initial test for the presence of bacteria. At block 204 the sample is incubated for about 3-4 hours. At block 205 the sample is tested for a second time. At block 206 a second determination is made whether to send the test ampoule to a central laboratory.

At the central laboratory, a master test system can be installed including the following components: a reader, single read and non-incubating or multi read auto-incubating; database and computer system with video conference capability; bar code reader; and test ampoules.

At the central laboratory, laboratory test data may be collected, up-loaded and reviewed.

For the culture-probable samples are sent to the central lab at block 207 together with a test data file at block 208. The test data file can be opened (at block 209) and reviewed (at block 210). Block 211 shows that the ampoule was previously inoculated (e.g., at block 201) and the negative screen protocol 2 is begun at block 212. At block 212 the sample is tested to determine whether a change has occurred during transportation (e.g., was the sample refrigerated during transportation to substantially prevent growth). If, at block 213, changes in the visible and infrared transmittance of the sample are outside predetermined ranges, or a combination thereof, then the test is concluded for that sample; note that the first test (e.g., block 201) is still valid. The predetermined range can be determined through experiment. The comparison at block 213 may result in a negative result or result in further incubation at block 214 for samples which have been verified to be within the predetermined range of the first read, followed by a second read at block 215 to confirm the presence of a culture at block 216. The central lab results of block 215 are compared to the first read at block 202. The computer aided analyst may then make speciation determinations for microbial species based on prior knowledge of test results for different species.

This laboratory protocol would have the added advantage of the first test-read data from the satellite collection lab at block 202. The use of the first read on the fresh sample versus a central lab first-read assists in false positive detection and the currently undetectable sample change (in conventional methods, e.g., streaking plates at a central lab) during the sample custody/transportation period.

The central laboratory review of test data from the satellite collection lab would be needed before reporting negatives. Any doubtful results would be confirmable or reviewed revisable by video conference and/or a re-reading of the retained samples.

For those samples deemed positive by the master system at the central laboratory, the contents of fresh positive test ampoules would be used to inoculate the speciation culture plate with log phase microbes. An ampoule neck-cutting process (breaking open the ampoule) can be applied to the test ampoule to allow for the speciation process.

The expanded satellite test system may exhibit one or more of the following:
Test inoculation in a consistent, timely manner—in keeping with published standards.
Improved elimination of false positive results.
Enhanced standardization and control of positive threshold determination.
Complete and instantaneous sample custody tracking.
Complete and instantaneous test data recording and review.
Significantly shorter time for both negative and positive UTI results reporting.
Substantially expanded control of the UTI culture system by central laboratory personnel.
Substantially more central bio-laboratory time to work on positive ID issues.

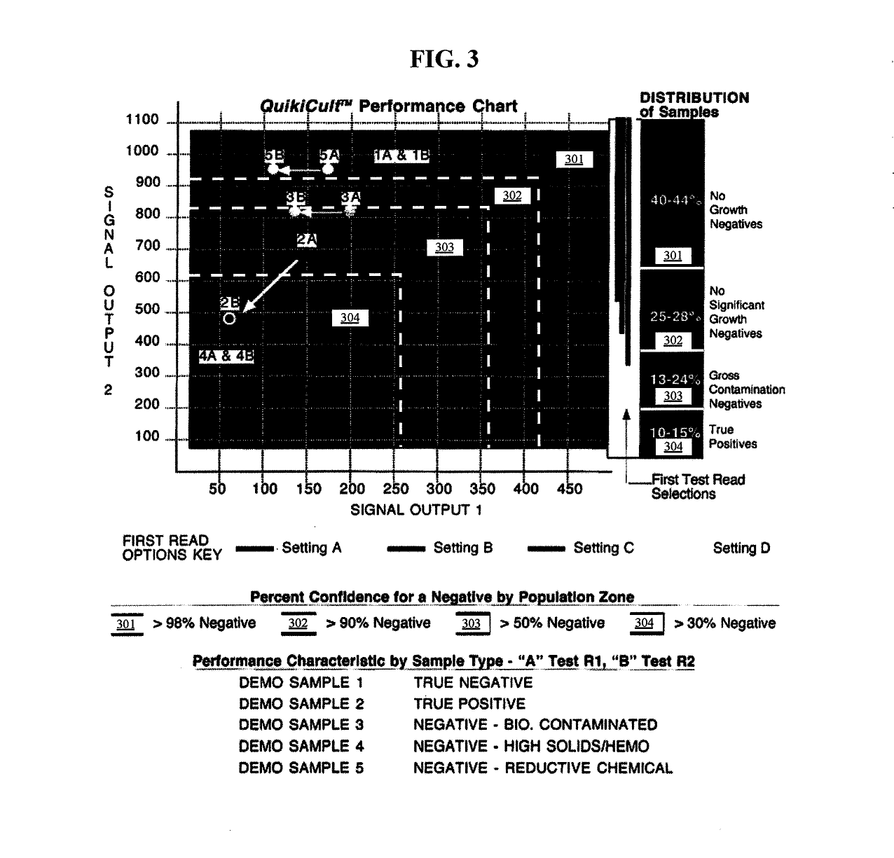

FIG. 3 is a graph of performance characteristics of exemplary implementations according to embodiments of the present disclosure. The graph includes population zones 301-304, which correspond to respective distribution and percent confidence values. In FIG. 3 zone 301 corresponds to a confidence of greater than 98% negative, these samples comprising about 40-44% no growth negatives; zone 302 corresponds to a confidence of greater than 90% for a negative (up to 98%), these samples comprising about 25-28% no significant growth negative; zone 303 corresponds to a confidence of greater than 50% negative (up to 90%), these samples comprising about 13-24% gross contamination negatives; and zone 304 corresponds to a confidence of greater than 30% negative (up to 50%), these samples comprising 10-15% true positives.

As shown in FIG. 3, sample populations 1-5 were observed during two tests. Changes in the sample populations where tracked, e.g., see population 1 shown as 1A and 1B, corresponding to the two tests.

It should be noted that thresholds (e.g., see FIG. 3 wherein 98% of the samples in the area above the section 301 are negative) for eliminating negative samples may be adjusted according to an application.

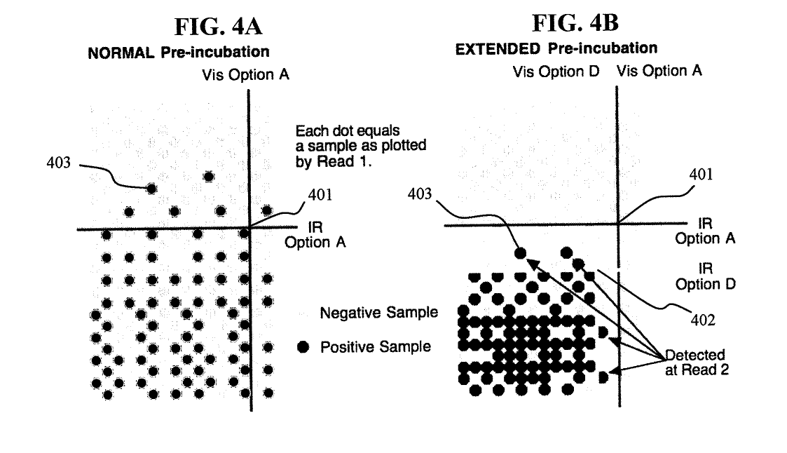

Referring to FIGS. 4A-B and the incubation period; pre-incubation after sample storage (e.g., for transport to a central laboratory) returns the sample to its original chemical and biological state. By extending the pre-incubation as shown in FIG. 4B, positive samples grow vigorously and thus lower their Vis/IR coordinate. For example, sample 403 in FIG. 4A migrates to a lower Vis/IR coordinate in FIG. 4B. The negatives and lag contaminants do not change their locus. The combined effect is to cause the positives to separate from negatives when plotted for Vis/IR coordinates. As compared to the criteria 401, this separation of positives from negatives can be used to enhance the effectiveness of the Read 1 selection of non-threshold negative samples through the use of optional criteria 402 for detecting positive samples. In a test device, the criteria may be selected manually or automatically, for example, if the test device detects an extended pre-incubation, the test methodology may be changed to an optional criteria.

The extended pre-incubation period also allows the confirming Read 2 to be made sooner as positives have less distance to vector to demonstrate positivity. FIGS. 5A-B demonstrate the effect of extended pre-incubation.

The period of pre-incubation can be varied according to the age of the same; for example, for a sample less than about 3 hours old, a pre-incubation of about 10-15 minutes can be used, for a sample about 4-12 hours old, a pre-incubation of 1 hours can be used, for a sample older than about 12 hours, a pre-incubation of about 1.5 hours can be used.

Referring to the negative screen, FIGS. 5A-B show graphs determined for determining negative samples for populations less than about 12 hours old (FIG. 5A) and for populations older than 12 hours (FIG. 5B).

According to an embodiment of the present disclosure, a grid map is created that segments visible and IR readings into sections, for example, 4 quadrants (QUAD 1-4), and a determination of positive/negative may be made according to an observations plot. For example, a particular value for each of visible and IR is optimized for the determination. For example, FIG. 5A shows positive and negative samples, wherein samples above about 900 nm (501) in the infrared (QUADS 1-2) tend to be negative and the lower left quadrant (QUAD 3) tend to be positive.

FIG. 5B is a graph of samples which have not be pre-incubated for an extended time. As compared to FIG. 5A, the samples have migrated to the upper left, potentially increasing the number of false negatives. Through the use of a pre-incubation protocol as described herein, the samples may be returned to an original state before testing.

Referring to FIG. 6, a first reading of a sample (e.g., light transmission through the sample at one or more wavelengths) is determined at block 601. If the reading is a first reading for the sample at block 602, the reading is compared to a first read index at block 603. A first read probability is determined according to the reading and the first read index at block 604. The first read probability gives either a positive or a negative result for the sample and negative screen data is recorded at block 605. The positive or negative result is associated with the sample. A second reading is determined at block 606 at a predetermined time after the first reading. The reading is compared to a second read index at block 607. A second read probability is determined according to the reading and the second read index at block 607. The second read probability gives either a positive or a negative result for the sample while screening for negative samples using the negative screen data from block 605 at block 608. According to the result (e.g., positive or negative) the sample is may be handled separately; for positive samples, values of the first and second readings are compared to a species and life phase index to determine a species and life phase of a bacteria in the sample at block 609. The results, e.g., that a sample is negative or that a sample is positive and is associated with a certain species having a certain life phase, are written to a file at block 610. It is determined whether an end of a batch of samples has been reached at block 611.

It is to be understood that the present invention may be implemented in various forms of hardware, software, firmware, special purpose processors, or a combination thereof. In one embodiment, the present invention may be implemented in software as an application program tangibly embodied on a program storage device. The application program may be uploaded to, and executed by, a machine comprising any suitable architecture.

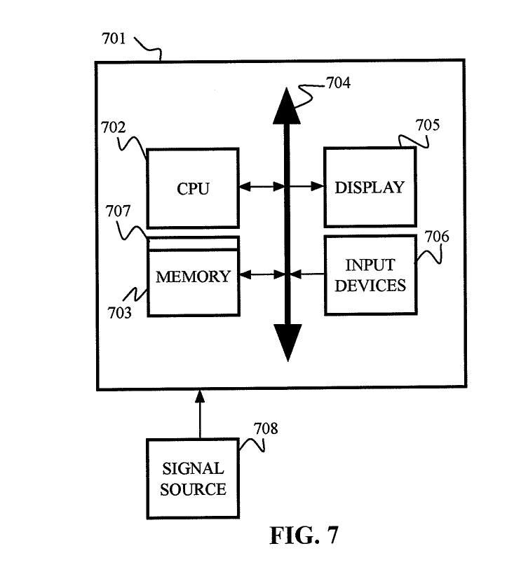

Referring to FIG. 7, according to an embodiment of the present invention, a computer system 701 detecting and analyzing microbial activity, inter alia, a central processing unit (CPU) 702, a memory 703 and an input/output (I/O) interface 704. The computer system 701 is generally coupled through the I/O interface 704 to a display 705 and various input devices 706 such as a mouse and keyboard. The support circuits can include circuits such as cache, power supplies, clock circuits, and a communications bus. The memory 703 can include random access memory (RAM), read only memory (ROM), disk drive, tape drive, or a combination thereof. Embodiments of the present disclosure can be implemented as a routine 607 that is stored in memory 703 and executed by the CPU 702 to process the signal from the signal source 708, e.g., spectrophotometer VIS/IR data. As such, the computer system 701 is a general-purpose computer system that becomes a specific-purpose computer system when executing the routine 707 of the present disclosure.

The computer platform 701 also includes an operating system and micro instruction code. The various processes and functions described herein may either be part of the micro instruction code, or part of the application program (or a combination thereof) which is executed via the operating system. In addition, various other peripheral devices may be connected to the computer platform such as an additional data storage device and a printing device.

It is to be further understood that, because some of the constituent system components and methods depicted in the accompanying figures may be implemented in software, the actual connections between the system components (or the processes) may differ depending upon the manner in which the present disclosure is programmed. Given the teachings of the present disclosure provided herein, one of ordinary skill in the related art will be able to contemplate these and similar implementations or configurations of the present disclosure.

Having described embodiments for a system and method for detecting and analyzing microbial activity, it is noted that modifications and variations can be made by persons skilled in the art in light of the above teachings. It is therefore to be understood that changes may be made in the particular embodiments of the invention disclosed which are within the scope and spirit of the disclosure.

What is claimed is:

1. A method for testing a microbial population including a negative screen comprising:
inoculating a plurality of test ampoules with respective samples;
performing a first light transmittance test on the test ampoules;
recording first test data;
performing a second light transmittance test on the test ampoules;
recording second test data;
detecting negative samples based on the first test data and the second test data; and
reporting negative samples based on the first test data and the second test data.

2. The method of claim 1, further comprising:
refrigerating the samples; and
performing a pre-incubation of the samples before one of the first test and the second test.

3. The method of claim 2, wherein the pre-incubation has a duration selected to correspond to an age of the samples.

4. The method of claim 3, wherein the duration of the pre-incubation is about 10 to 90 minutes.

5. The method of claim 1, wherein the samples are incubated during the second test for a constant time.

6. The method of claim 5, wherein the constant time is about 60 minutes.

7. A method for testing a microbial population including a false positive screen comprising:
inoculating a plurality of test ampoules with respective samples;
performing a first light transmittance test on the test ampoules;
recording first test data;
performing a second light transmittance test on the test ampoules;
recording second test data;
detecting samples positive for the presence of the microbial population based on the first test data and the second test data; and
reporting the samples positive for the presence of the microbial population based on the first test data and the second test data.

8. The method of claim 7, refrigerating the samples; and performing a pre-incubation of the samples before one of the first test and the second test.

9. The method of claim 8, wherein the pre-incubation has a duration selected to correspond to an age of the samples.

10. The method of claim 9, wherein the duration of the pre-incubation is about 10 to 90 minutes.

11. The method of claim 7, wherein the samples are incubated during the second test for a constant time.

12. The method of claim 11, wherein the constant time is about 60 minutes.
